(19) United States
(12) Patent Application Publication
(10) Pub. No.: US 2025/0387003 A1
(43) Pub. Date: Dec. 25, 2025

(54) MEDICAL SYSTEM, CONTROL DEVICE, AND CONTROL METHOD

(71) Applicant: OLYMPUS MEDICAL SYSTEMS CORP., Tokyo (JP)

(72) Inventors: Shota SAWADA, Saitama (JP); Noriaki YAMANAKA, Tokyo (JP)

(73) Assignee: OLYMPUS MEDICAL SYSTEMS CORP., Tokyo (JP)

(21) Appl. No.: 19/089,477

(22) Filed: Mar. 25, 2025

Related U.S. Application Data
(60) Provisional application No. 63/663,437, filed on Jun. 24, 2024.

(51) Int. Cl. A61B 1/00; A61B 1/005; A61B 90/00; G16H 40/63

(52) U.S. Cl. CPC A61B 1/00006; A61B 1/0002; A61B 1/0057; A61B 90/06; G16H 40/63; A61B 2090/064

(57) ABSTRACT

The present invention provides a control device that controls a driving device connected to a proximal end of a flexible medical manipulator. The driving device drives at least one wire for bending a bending section at a distal end of the medical manipulator. Tension of the wire is measured at the proximal end. The control device includes at least one processor. In a state where a bend angle of the bending section is set to a certain angle, the processor is configured to control the driving device to pull or loosen the wire within a range in which the bend angle is maintained at the certain angle to detect an external force acting on the bending section based on a change in the tension of the wire.

### CROSS-REFERENCE TO RELATED APPLICATIONS

This application is based upon and claims the benefit of priority from U.S. Provisional Application No. 63/663,437, filed on Jun. 24, 2024; the entire contents of which are incorporated herein by reference.

### TECHNICAL FIELD

The present invention relates to medical systems, control devices, and control methods.

### BACKGROUND ART

A known motorized endoscope in the related art bends the distal end of an insertion section by driving a wire (e.g., see Patent Literature 1). This endoscope uses a tension sensor disposed at the proximal end of a flexible section of the endoscope to detect a load applied to a bending section at the distal end, and drives the wire in a direction that reduces the load, thereby avoiding an excessive load applied to biological tissue from the bending section.

### CITATION LIST

### Patent Literature

{PTL 1}
The Publication of Japanese Patent No. 6157063

### SUMMARY OF INVENTION

An aspect of the present invention provides a control device that controls a driving device connected to a proximal end of a flexible medical manipulator. The driving device drives at least one wire for bending a bending section at a distal end of the medical manipulator. Tension of the wire is measured at the proximal end. The control device includes at least one processor. In a state where a bend angle of the bending section is set to a certain angle, the processor is configured to control the driving device to pull or loosen the wire within a range in which the bend angle is maintained at the certain angle to detect an external force acting on the bending section based on a change in the tension of the wire.

Another aspect of the present invention provides a medical system including: a flexible medical manipulator that includes at least one wire for bending a bending section at a distal end of the medical manipulator and a sensor that measures tension of the wire at a proximal end of the medical manipulator; a driving device that is connected to the proximal end of the medical manipulator and that drives the medical manipulator; and a control device that controls the driving device. The control device includes at least one processor. In a state where a bend angle of the bending section is set to a certain angle, the processor is configured to control the driving device to pull or loosen the wire within a range in which the bend angle is maintained at the certain angle so that the sensor detects an external force acting on the bending section based on a change in the tension of the wire.

Another aspect of the present invention provides a control method for driving at least one wire for bending a bending section at a distal end of a flexible medical manipulator. The control method includes: measuring tension of the wire at a proximal end of the medical manipulator; and performing control to pull or loosen the wire within a range in which a bend angle of the bending section is maintained at a certain angle to detect an external force acting on the bending section based on a change in the tension of the wire from a state where the bend angle is set to the certain angle.

### BRIEF DESCRIPTION OF DRAWINGS

FIG. 1 is an overall configuration diagram illustrating the configuration of a medical system according to a first embodiment.

FIG. 2 illustrates an insertion section of an endoscope of the medical system in FIG. 1.

FIG. 3 is a cross-sectional view illustrating the insertion section of the endoscope of the medical system in FIG. 1.

FIG. 4 is a perspective view illustrating a first attachable-detachable section prior to being attached to a driving device of the medical system in FIG. 1.

FIG. 5A illustrates a wire attachable-detachable section, prior to being attached to the driving device, and a wire driver in the medical system in FIG. 1.

FIG. 5B illustrates the wire attachable-detachable section, attached to the driving device, and the wire driver in the medical system in FIG. 1.

FIG. 6 is a block diagram illustrating a control device according to this embodiment.

FIG. 7 illustrates the relationship among the drive amount, the tension, and the bend angle of a wire.

FIG. 8A schematically illustrates the shape of the endoscope in a state where a bending section is bent in one direction by pulling one of wires.

FIG. 8B illustrates the distribution of tension at each position of the one wire in the longitudinal direction in FIG. 8A.

FIG. 9 is a flowchart illustrating a control method executed by the control device in FIG. 6.

FIG. 10A schematically illustrates the shape of the endoscope in a state where the one wire is loosened from the state in FIG. 8A within a range in which the bend angle of the bending section is maintained.

FIG. 10B illustrates the distribution of tension at each position of the wire in the longitudinal direction in FIG. 10A.

FIG. 11A schematically illustrates the shape of the endoscope and explains a state where an external force acts in a second direction in the distribution of tension of the wire in FIG. 11B.

FIG. 11B illustrates the distribution of tension at each position of the wire in the longitudinal direction in FIG. 11A.

FIG. 12A schematically illustrates the shape of the endoscope when an external force acting in a first direction is detected from the state in FIG. 10A.

FIG. 12B illustrates the distribution of tension at each position of the wire in the longitudinal direction in the state in FIG. 12A.

FIG. 13A schematically illustrates the shape of the endoscope and explains a state where an external force acts in the first direction in the distribution of tension of the wire in FIG. 12B.

FIG. 13B illustrates the distribution of tension at each position of the wire in the longitudinal direction in FIG. 13A.

FIG. 14A schematically illustrates the shape of the endoscope in a state where the one wire is loosened from the state in FIG. 8A to an intermediate point of the range in which the bend angle of the bending section is maintained.

FIG. 14B illustrates the distribution of tension at each position of the wire in the longitudinal direction in FIG. 14A.

FIG. 15A illustrates a wire attachable-detachable section, prior to being attached to the driving device, and a wire driver in the medical system according to a second embodiment of the present invention.

FIG. 15B illustrates the wire attachable-detachable section, attached to the driving device, and the wire driver in the medical system in FIG. 15A.

FIG. 16A schematically illustrates the shape of the endoscope in a state where a second wire is pulled within the range in which the bend angle of the bending section is maintained from the state in FIG. 8A in which a first wire is pulled.

FIG. 16B illustrates the distribution of tension at each position of the wire in the longitudinal direction in FIG. 16A.

FIG. 17A schematically illustrates the shape of the endoscope and explains a state where an external force acts in the second direction in the distribution of tension of the wire in FIG. 16B.

FIG. 17B illustrates the distribution of tension at each position of the wire in the longitudinal direction in FIG. 17A.

FIG. 18A schematically illustrates the shape of the endoscope when an external force acting in the first direction is detected from the state in FIG. 16A.

FIG. 18B illustrates the distribution of tension at each position of the wire in the longitudinal direction in FIG. 18A.

FIG. 19A schematically illustrates the shape of the endoscope and explains a state where an external force acts in the first direction in the distribution of tension of the wire in FIG. 18B.

FIG. 19B illustrates the distribution of tension at each position of the wire in the longitudinal direction in FIG. 19A.

### DESCRIPTION OF EMBODIMENTS

A medical system 100, a control device 40, and a control method according to the first embodiment of the present invention will now be described with reference to the drawings.

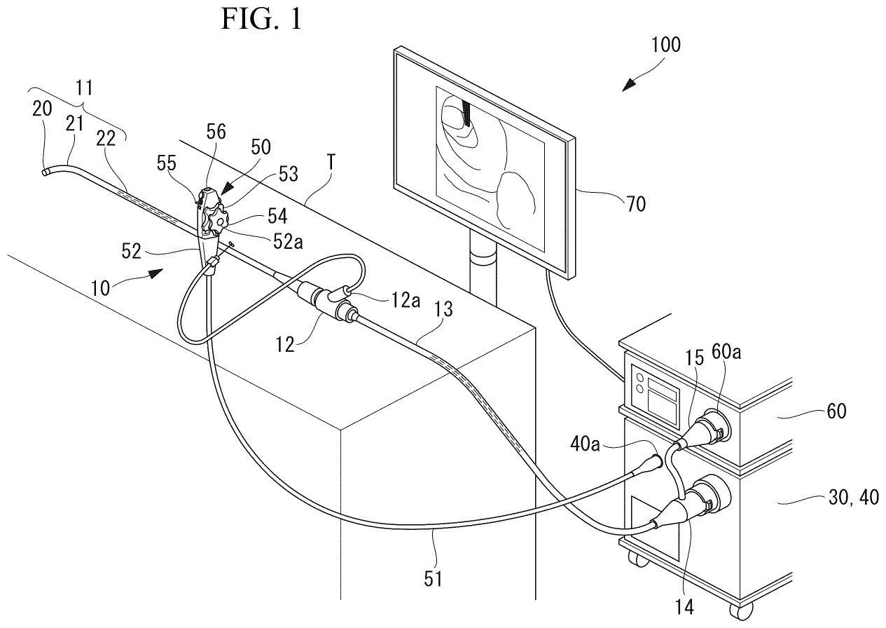

As shown in FIG. 1, the medical system 100 according to this embodiment is a system for observing and treating the inside of the body of a patient lying on a surgical table T. The medical system 100 includes a medical manipulator 10, a driving device 30, and the control device 40. In this embodiment, the driving device 30 and the control device 40 are accommodated within the same cabinet. The medical system 100 further includes a manipulation device 50, a video control device 60, and a display device 70.

The medical manipulator 10 is a flexible endoscope (referred to as “endoscope 10” hereinafter) to be inserted into the patient's lumen. An endoscopic image acquired by the endoscope 10 is input to the display device 70 via the video control device 60, so as to be displayed on the display device 70.

The manipulation device 50 is connected to an adapter 40a of the control device 40 via a manipulation cable 51. An operation input received by the manipulation device 50 is input to the control device 40 from the manipulation device 50. The control device 40 controls the driving device 30 based on the operation input received by the manipulation device 50. Accordingly, the endoscope 10 is operated in accordance with the operation input.

The endoscope 10 is detachably connected to the driving device 30. In the description hereinafter, the side of the endoscope 10 to be inserted into the patient's lumen will be referred to as the distal end, whereas the side to be attached to the driving device 30 will be referred to as the proximal end.

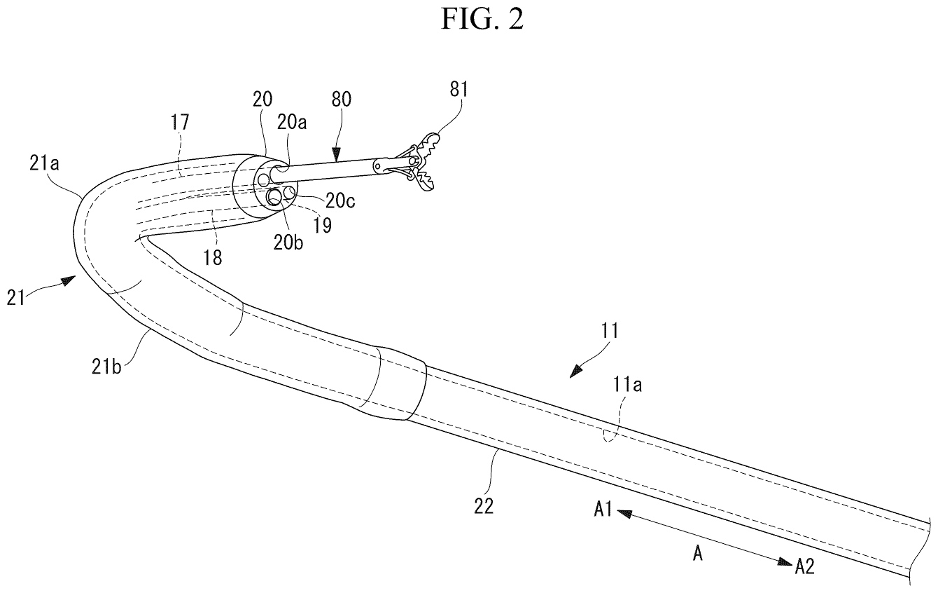

As shown in FIG. 1, the endoscope 10 includes an insertion section 11, a coupling section 12, an external flexible section 13, a first attachable-detachable section 14, and a second attachable-detachable section 15, in that order from the distal end. The insertion section 11 is a long flexible member. As shown in FIG. 2, the insertion section 11 has an internal path 11a therein.

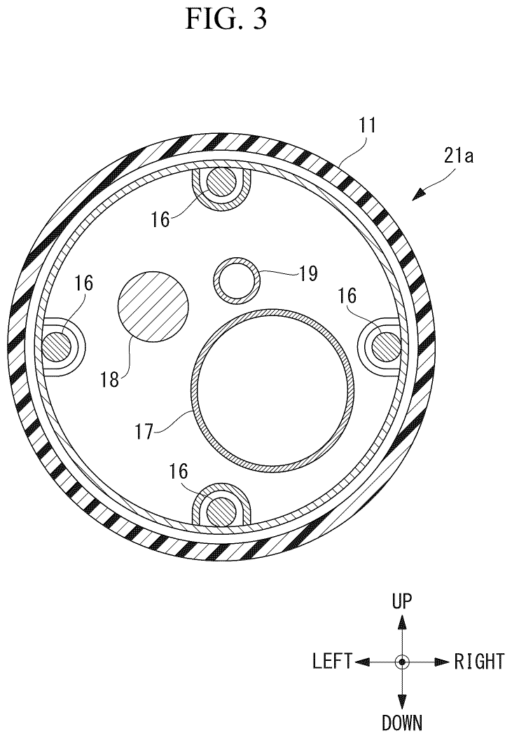

The internal path 11a of the endoscope 10 extends in a longitudinal direction A of the endoscope 10 from the distal end of the insertion section 11 to the proximal end of the first attachable-detachable section 14. As shown in FIG. 3, the internal path 11a accommodates, for example, wires 16, a channel tube 17, a light guide 18, and an imaging cable 19 that are to be described later.

The insertion section 11 has a distal end section 20, a bending section 21, and an internal flexible section 22, in that order from the distal end.

As shown in FIG. 2, the distal end section 20 includes an opening 20a communicating with the channel tube 17, an illuminating unit 20b, and an imaging unit 20c. A treatment unit 81, such as grasping forceps, provided at the distal end of a treatment tool 80 extending through a channel within the channel tube 17 protrudes and retracts through the opening 20a. The light guide 18 is connected to the illuminating unit 20b, and the imaging cable 19 is connected to the imaging unit 20c.

The bending section 21 has a first bending section 21a and a second bending section 21b provided at the proximal end of the first bending section 21a. Each of the first bending section 21a and the second bending section 21b is bendable upward, downward, leftward, and rightward.

As shown in FIG. 3, the first bending section 21a is connected to four wires 16 for bending the first bending section 21a upward, downward, leftward, and rightward, respectively. Similarly, the second bending section 21b is also connected to other four wires 16 for bending the second bending section 21b upward, downward, leftward, and rightward, respectively. The first bending section 21a and the second bending section 21b are independently bendable in different directions.

The coupling section 12 couples the internal flexible section 22 of the insertion section 11 and the external flexible section 13 to each other. The coupling section 12 is provided with an insertion port 12a for inserting the treatment tool 80 into the channel tube 17 in the internal path 11a.

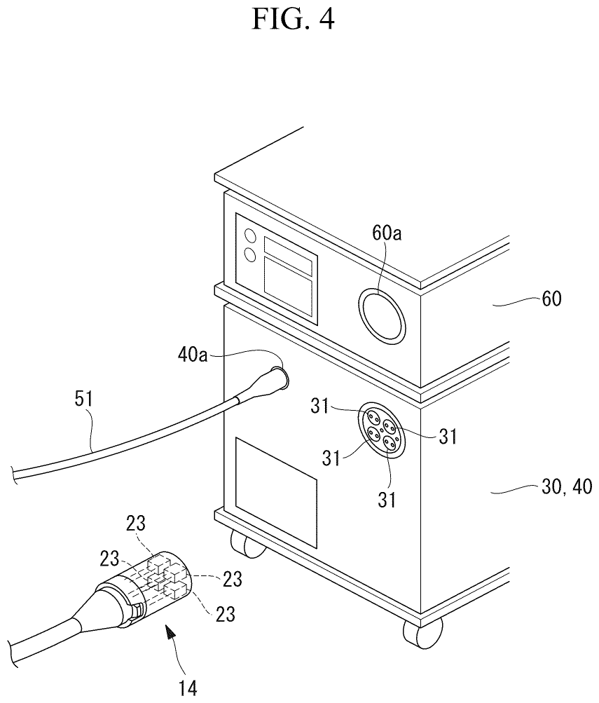

As shown in FIG. 4, the first attachable-detachable section 14 has four wire attachable-detachable sections 23 as a mechanism for detachably connecting the wires 16 to the driving device 30. Each wire attachable-detachable section 23 is provided at the proximal end of a pair of wires 16 and attaches and detaches the wires 16 to and from the driving device 30. For example, the four wire attachable-detachable sections 23 respectively attach and detach a pair of wires 16 for bending the first bending section 21a upward and downward, a pair of wires 16 for bending the first bending section 21a leftward and rightward, a pair of wires 16 for bending the second bending section 21b upward and downward, and a pair of wires 16 for bending the second bending section 21b leftward and rightward to and from the driving device 30.

The second attachable-detachable section 15 is detachably connected to an adapter 60a of the video control device 60. The light guide 18 and the imaging cable 19 are connected to the video control device 60 via the second attachable-detachable section 15.

The driving device 30 is connected to a power source (not shown) and operates in accordance with electric power supplied from the power source. As shown in FIG. 4, the driving device 30 has four wire drivers 31 as a mechanism for driving the wires 16. By being connected to the first attachable-detachable section 14, the four wire drivers 31 are respectively coupled to the four wire attachable-detachable sections 23 and can each drive the corresponding pair of wires 16. For example, the four wire drivers 31 respectively drive the pair of wires 16 for bending the first bending section 21a upward and downward, the pair of wires 16 for bending the first bending section 21a leftward and rightward, the pair of wires 16 for bending the second bending section 21b upward and downward, and the pair of wires 16 for bending the second bending section 21b leftward and rightward.

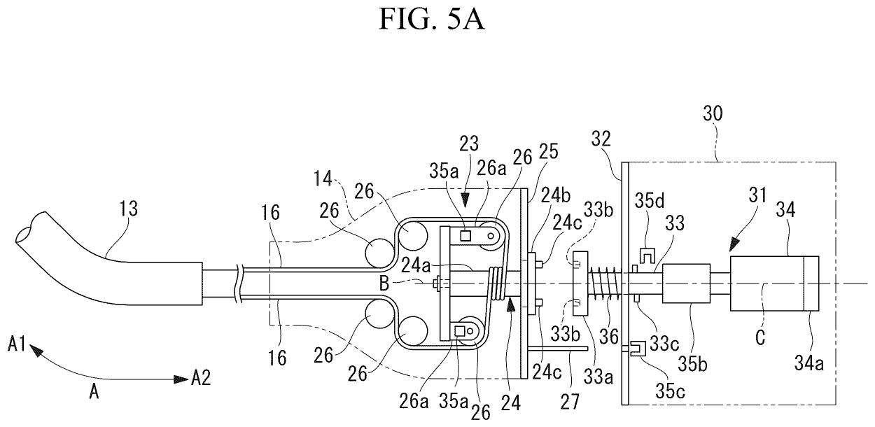

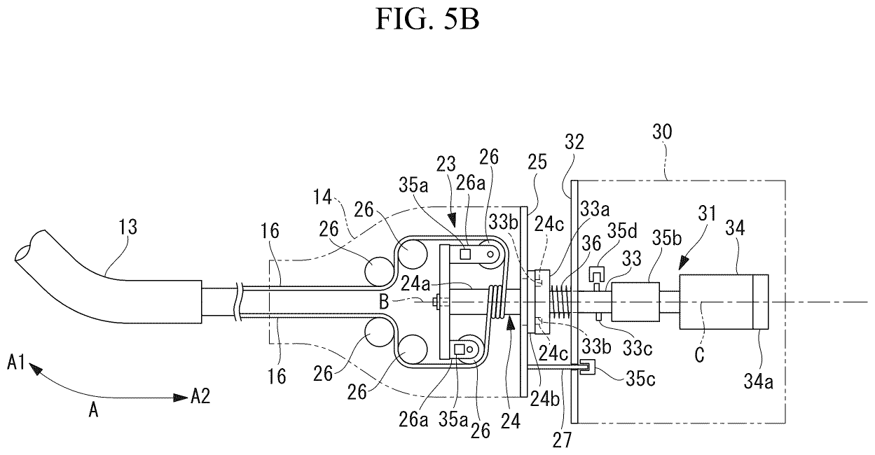

FIGS. 5A and 5B illustrate the configuration of each wire attachable-detachable section 23 and each wire driver 31. FIG. 5A illustrates the wire attachable-detachable section 23 and the wire driver 31 in a state where they are separated from each other, and FIG. 5B illustrates the wire attachable-detachable section 23 and the wire driver 31 in a state where they are connected to each other.

For example, FIGS. 5A and 5B illustrate the wire attachable-detachable section 23, including the pair of wires 16 for bending the first bending section 21a upward and downward, and the wire driver 31. Since the remaining wire attachable-detachable sections 23 and the remaining wire drivers 31 also have the configuration in FIGS. 5A and 5B, redundant descriptions will be omitted.

Each wire attachable-detachable section 23 has a rotating drum 24 and a support member 25 for supporting the rotating drum 24. The support member 25 is to be secured to a support member 32 of the wire driver 31 in a state where the wire attachable-detachable section 23 is coupled to the wire driver 31.

The rotating drum 24 is supported by the support member 25 in such a manner as to be rotatable around a rotation axis B extending in the longitudinal direction A of the insertion section 11. The rotating drum 24 includes a winding pulley 24a disposed coaxially with the rotation axis B and a coupling 24b fixed to the winding pulley 24a.

The pair of wires 16 are guided to the winding pulley 24a via at least one pulley 26 that guides the wires 16, and are wound around the outer peripheral surface of the winding pulley 24a. The winding pulley 24a rotates around the rotation axis B so as to pull or feed the pair of wires 16.

The coupling 24b is a disk member that is fixed to the proximal end of the winding pulley 24a and that is disposed coaxially with the rotation axis B, and is exposed at the proximal end of the wire attachable-detachable section 23. The surface at the proximal end of the coupling 24b is provided with two engagement protrusions 24c at opposite sides of the rotation axis B.

Each wire attachable-detachable section 23 includes a dog 27 that is provided on the support member 25 and that is for detecting whether the wire attachable-detachable section 23 is attached to or detached from the wire driver 31.

The dog 27 is a member protruding outward of the wire attachable-detachable section 23 from the support member 25 and exposed at the proximal end of the wire attachable-detachable section 23, and is, for example, a pin-shaped member extending parallel to the rotation axis B. As shown in FIG. 5B, in a state where the wire attachable-detachable section 23 is connected to the wire driver 31, the dog 27 extends through the support member 32 of the wire driver 31 and is inserted into the wire driver 31.

The wire driver 31 has a shaft 33, a motor 34 connected to the shaft 33, and the support member 32 that rotatably supports the shaft 33.

The shaft 33 is supported by the support member 32 in such a manner as to be rotatable around a rotation axis C and extendable and retractable in the longitudinal direction A. The rotation axis C is the center axis of the shaft 33 and is aligned with the rotation axis B of the rotating drum 24 in a state where the first attachable-detachable section 14 is connected to the driving device 30.

The motor 34 is, for example, a direct-current motor. The motor 34 generates a rotational force as a driving force in accordance with the electric power supplied from the power source, and rotates the shaft 33 around the rotation axis C. In the wire driver 31, the motor 34 is provided with an encoder 34a that detects the rotational speed and the rotational angle of the motor 34. The encoder 34a is connected to the proximal end of the motor 34.

The wire driver 31 has a coupling 33a as a mechanism that is provided at the shaft 33 and that couples the motor 34 to the rotating drum 24.

The coupling 33a is a disk member that is fixed to the distal end of the shaft 33 and that is disposed coaxially with the rotation axis C, and rotates integrally with the shaft 33. The coupling 33a is exposed at the distal end of the wire driver 31. The surface at the distal end of the coupling 33a is provided with two engagement recesses 33b at opposite sides of the rotation axis C.

As shown in FIG. 5B, with the engagement between the engagement protrusions 24c and the engagement recesses 33b, the coupling 24b and the coupling 33a become coupled to each other, whereby the motor 34 becomes coupled to the wires 16 via the rotating drum 24. In this state, the rotating drum 24, the coupling 24b, the coupling 33a, and the shaft 33 are integrally rotatable around the rotation axes B and C. Therefore, the rotational force (driving force) generated by the motor 34 is transmitted as a force acting in the longitudinal direction A to the wires 16 via the rotating drum 24.

When the motor 34 is rotated in one direction around the rotation axis C, the rotating drum 24 is rotated in one direction around the rotation axis B. As a result, for example, one (first wire) of the wires 16 of the pair disposed at opposite sides in the up-down direction is pulled, whereas the other (second wire) is loosened. In contrast, when the motor 34 is rotationally driven in the other direction, the wires 16 that are pulled and loosened are interchanged. Thus, the rotational driving direction of the motor 34 is switched, so that the bending section 21 can be bent either upward or downward. The same applies to the leftward and rightward directions.

The medical system 100 further includes a tension sensor (sensor) 35a, a torque sensor 35b, an attachment-detachment sensor 35c, a coupling sensor 35d, and an electric-current sensor (not shown).

The tension sensor 35a is provided for each of the four wire attachable-detachable sections 23. The torque sensor 35b, the attachment-detachment sensor 35c, the coupling sensor 35d, and the electric-current sensor are provided for each of the four wire drivers 31. The sensors 35a, 35b, 35c, and 35d are connected to the control device 40. An output from each of the sensors 35a, 35b, 35c, and 35d is successively transmitted to the control device 40.

The tension sensor 35a is provided for each wire 16. For example, the tension sensor 35a is constituted of a distortion sensor installed in a support pillar 26a of the pulley 26 and measures the tension of the wire 16 in accordance with distortion of the support pillar 26a. By using the tension sensor 35a to measure the tension for every drive amount of the wire 16, information related to a change in the tension of the wire 16 can be acquired.

The torque sensor 35b is provided for each motor 34 and detects the torque of the motor 34. For example, the torque sensor 35b is attached to the shaft 33 and detects torque acting around the rotation axis C as the torque of the motor 34.

The attachment-detachment sensor 35c detects whether the wire attachable-detachable section 23 is attached to or detached from the wire driver 31. When the wire attachable-detachable section 23 is connected to the wire driver 31, the attachment-detachment sensor 35c engages with the dog 27 extending through the support member 32 and inserted into the wire driver 31. The attachment-detachment sensor 35c has, for example, an optical sensor that detects that the dog 27 is in contact therewith or is in close vicinity thereof, and uses the optical sensor to detect engagement with the dog 27.

The coupling sensor 35d is provided for each motor 34. The coupling sensor 35d detects engagement between the coupling 24b and the coupling 33a based on shifting of the shaft 33, so as to detect that the motor 34 is coupled to the wires 16.

As shown in FIG. 5B, the coupling 33a is pressed by the coupling 24b, so as to move toward a proximal end A2 together with the shaft 33. The coupling sensor 35d has, for example, an optical sensor that detects that a dog 33c provided on the shaft 33 is approaching, and detects engagement between the coupling 24b and the coupling 33a based on the approaching of the dog 33c.

The coupling 33a is biased toward a distal end A1 by an elastic member 36, such as a compression spring, disposed between the coupling 33a and the support member 32. As shown in FIG. 5A, in a state where the wire attachable-detachable section 23 and the wire driver 31 are separated from each other, the coupling 33a moves toward the distal end A1 together with the shaft 33 in accordance with the biasing force of the elastic member 36, so that the dog 33c is disposed at a position located away from the coupling sensor 35d. In this state, the coupling sensor 35d does not detect engagement between the coupling 24b and the coupling 33a. The electric-current sensor is provided for each motor 34, and detects an electric current flowing to the motor 34.

The manipulation device 50 is used by an operator, such as a surgeon, for inputting an operation for driving the endoscope 10. The received operation input is transmitted to the control device 40 via the manipulation cable 51.

As shown in FIG. 1, the manipulation device 50 includes a main body 52, a first angle knob 53, and a second angle knob 54.

The main body 52 has, for example, a shape that can be held with the left hand of the operator, such as a surgeon.

The first angle knob 53 and the second angle knob 54 are attached to the main body 52 in such a manner as to rotate around the same rotation axis 52a. For example, when the operator uses the right hand to rotate the first angle knob 53, the wires 16 for bending the bending section 21 upward and downward are driven. For example, when the second angle knob 54 is rotated, the wires 16 for bending the bending section 21 leftward and rightward are driven.

The control device 40 acquires the operation input from the manipulation device 50 via the adapter 40a. The control device 40 controls the driving device 30 based on the acquired operation input.

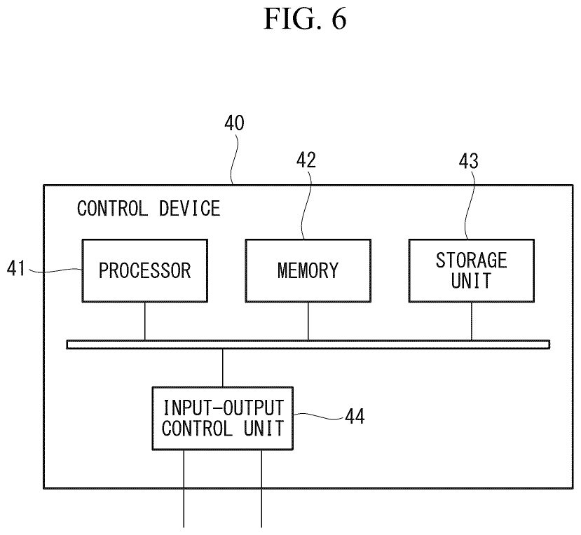

As shown in FIG. 6, the control device 40 is a program-executable computer that includes at least one processor 41, at least one memory 42, a storage unit 43 capable of storing a program and data, and an input-output control unit (interface) 44.

The storage unit 43 is a non-transitory nonvolatile recording medium that stores programs and required data, and is, for example, a ROM or a hard disk. The functions, to be described later, of the control device 40 are implemented as a result of a control program stored in the storage unit 43 being loaded to the memory 42 and being executed by the processor 41. At least one or more of the functions of the control device 40 may be implemented by a dedicated logical circuit.

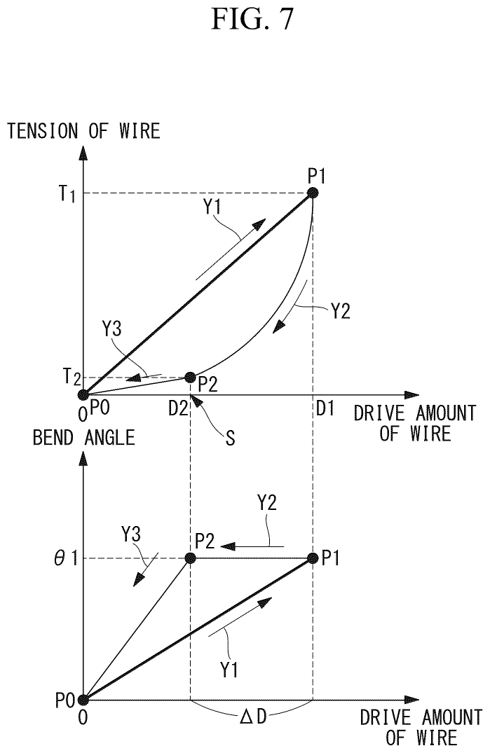

The memory 42 has stored therein a model capable of outputting the drive amount of each wire 16 that can be pulled or loosened within a range in which a bend angle θ is maintained by inputting the drive amount of the wire 16 and the tension of the wire 16. This model may be capable of outputting the drive amount of each wire 16 that can be pulled or loosened within the range in which the bend angle θ is maintained by inputting the drive amount of the wire 16 or the tension of the wire 16. As compared with a state where the bending section 21 is straight, the bend angle is an angle changed when the bending section 21 is bent, assuming that the straight state is 0 degrees. Alternatively, it is possible to determine this from an imaging direction at the distal end of the bending section 21. Assuming that the imaging direction when the bending section 21 is straight is 0 degrees, the aforementioned angle is an angle between the imaging direction when the bending section 21 is straight and the imaging direction after the bending section 21 is bent. For example, the relationship among the drive amount of the wire 16, the tension of the wire 16, and the bend angle of the bending section 21 in the model is as shown in FIG. 7. Starting from an origin point where the drive amount of the wire 16 is zero, when the wire 16 is pulled, the tension and the bend angle of the wire 16 increase substantially linearly in proportion to the drive amount of the wire 16, as indicated by an arrow Y1.

After the bend angle reaches an arbitrary first angle θ1, when the bend angle is to be decreased by loosening the wire 16, the tension of the wire 16 decreases rapidly without being proportional to the drive amount of the wire 16 until the drive amount of the wire 16 decreases to a predetermined point S, as indicated by an arrow Y2.

Then, when the drive amount of the wire 16 decreases to the predetermined point S, the bend angle starts to decrease together with the decrease in the drive amount of the wire 16 at that point, as indicated by an arrow Y3. Then, the tension and the bend angle of the wire 16 start to decrease substantially linearly again in proportion to the drive amount of the wire 16.

Thus, the relationship between the drive amount and the tension of the wire 16 and the relationship between the drive amount and the bend angle of the wire 16 each form a hysteresis curve in which the path differs between when the wire 16 is pulled and when the wire 16 is loosened.

Specifically, the operator first rotates the first angle knob 53 or the second angle knob 54 in one direction, so as to cause the drive amount of one of the wires 16 to increase and to cause the bend angle of the bending section 21 to reach the first angle θ1. Subsequently, when the operator rotates the first angle knob 53 or the second angle knob 54 in the other direction to decrease the drive amount of the wire 16, the tension of the wire 16 greatly decreases while the bend angle is maintained without being changed. In other words, the wire 16 can be loosened in a range in which the bend angle of the bending section is maintained.

Therefore, with the use of the model stored in the memory 42, the drive amount (dead zone) of the wire 16 for loosening the wire 16 within the range in which the bend angle is maintained can be acquired by using the drive amount of the wire 16 and the tension of the wire 16 at each time point.

The input-output control unit 44 is connected to the driving device 30, the manipulation device 50, and the display device 70. Based on control of the processor 41, the input-output control unit 44 performs exchanging of data and exchanging of control signals with connected devices.

Next, the control method executed by the control device 40 according to this embodiment will be described below.

In the control device 40 according to this embodiment, the processor 41 acquires an operation input generated as a result of the operator, such as a surgeon, operating the first angle knob 53 or the second angle knob 54 of the manipulation device 50, and controls the driving device 30.

In detail, the processor 41 actuates any of the motors 34 corresponding to the operation input in the driving device 30, thereby pulling and loosening the wires 16. Accordingly, the bending section 21 can be bent in a desired direction by a desired bend angle.

The control device 40 receives a predetermined trigger so as to enter an external-force detection mode. For example, a situation where a timer (not shown) measures a predetermined time period lapsed from when the manipulation device 50 no longer receives an operation input is set as the trigger. Alternatively, a switching operation performed by the operator using a switch 55 or an audio sensor 56 provided in the manipulation device 50 may be set as the trigger.

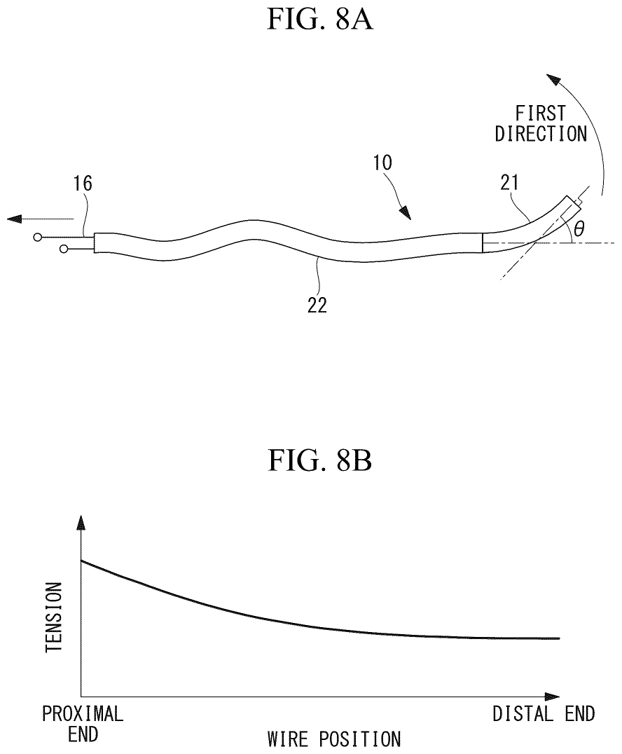

As one example, the following description relates to a case where a trigger is input to the processor 41 in a state where the operator operates the first angle knob 53 of the manipulation device 50 to increase the tension of one of the wires 16 and to bend the bending section 21 in a first direction to a first position P1 in FIG. 7 where the first angle θ1 is reached. In this case, the drive amount of the one wire 16 is a drive amount D1, and the tension thereof is a tension T1. An example of the shape of the endoscope 10 in this state is as shown in FIG. 8A, and the distribution of tension of the wire 16 in the lengthwise direction of the endoscope 10 is as shown in FIG. 8B.

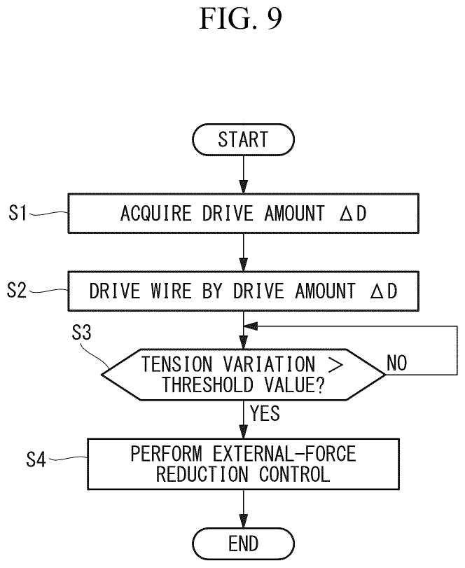

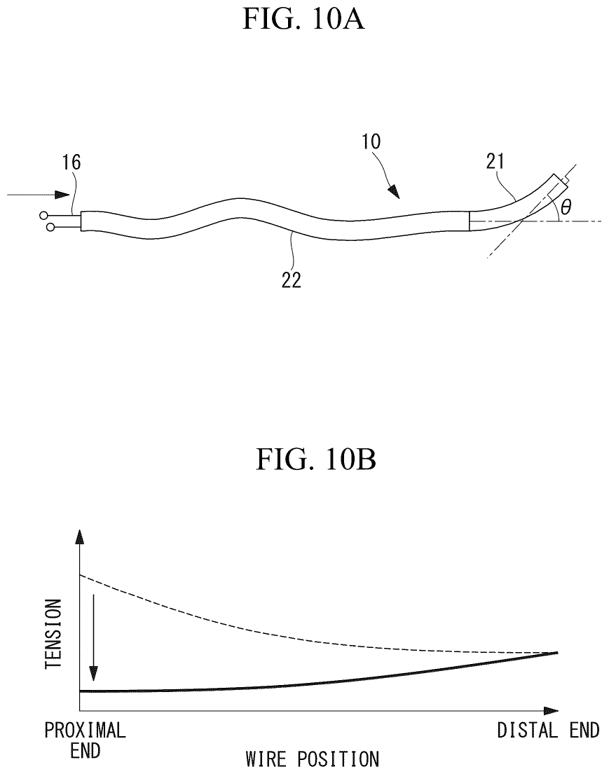

As shown in FIG. 9, the processor 41 inputs the drive amount D1 and the tension T1 of the one wire 16 into the model in the memory 42, so as to acquire a drive amount ΔD of the wire 16 corresponding to the dead zone (step S1). The processor 41 drives the motor 34 according to the acquired drive amount ΔD of the wire 16, so as to loosen the wire 16 (step S2). Accordingly, the bending section 21 moves to a second position P2 in FIG. 7. This is a state immediately before the bend angle of the bending section 21 starts to change from the first angle θ1. An example of the shape of the endoscope 10 in this state is as shown in FIG. 10A, and the distribution of tension of the wire 16 in the lengthwise direction of the endoscope 10 is as shown in FIG. 10B.

Depending on the input drive amount D1 and the input tension T1 of the wire 16, the acquired drive amount ΔD may be 0. An example is a case where the bending section 21 is loosened to a wire drive amount from the first position P1 to the second position P2 or less in FIG. 7 in response to an operation by the operator. In such a case, the distribution of tension of the wire 16 is already a preferable state to detect external force to the bending section 21 as shown in FIG. 10B. Therefore, even when a trigger is input to the processor 41, the processor 41 may sometimes need not to loosen the wire 16 in accordance with the operation of the motor 34.

If a wire is stationary in the distribution of tension in FIG. 8B, the tension acting toward the proximal end A2 is greater than the tension acting toward the distal end A1 at each position of the wire 16, and a stationary frictional force acts toward the distal end A1 to balance this out. In order to transmit an external force acting in a second direction on the distal end of the bending section 21 to the proximal end of the wire 16, the tensile relationship between the distal end and the proximal end of the wire 16 needs to be inverted. Specifically, even if an external force acts in the second direction to the distal end of the bending section 21 to decrease the bend angle in the distribution of tension in FIG. 8B, the distribution of tension at each position of the wire 16 is simply inverted gradually from the distal end toward the proximal end, thus requiring much high external force until the external force is detected by the tension sensor 35a at the proximal end of the wire 16.

On the other hand, if the wire 16 is stationary in the distribution of tension indicated with a solid line in FIG. 10B, the stationary frictional force acting toward the distal end A1 at each position of the wire 16 has vanished. Therefore, when an external force acts in the second direction onto the distal end of the bending section 21 to decrease the bend angle in this state, the external force is immediately transmitted to the tension sensor 35a at the proximal end of the wire 16, so that the change in the tension can be detected with high sensitivity.

According to this embodiment, in order to achieve the distribution of tension indicated with the solid line in FIG. 10B in which the stationary frictional force acting toward the distal end A1 has vanished, the wire 16 is loosened by the drive amount ΔD corresponding to the dead zone, so that the bend angle of the bending section 21 remains to be maintained at the first angle θ1. Therefore, the bent state of the bending section 21 of the endoscope 10 does not change, so that an endoscopic image displayed on the display device 70 does not change.

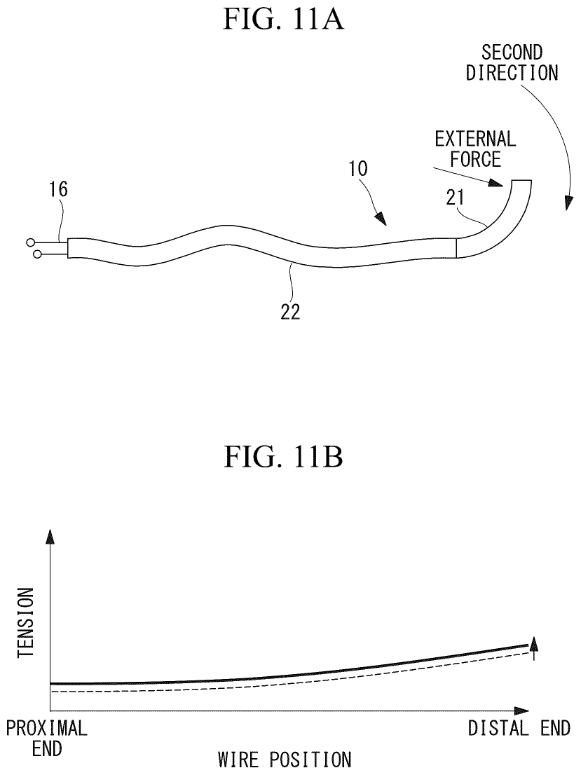

When the insertion section 11 is to be pulled out from the inside of the body in this state, if the distal end of the insertion section 11 comes into contact with the inner wall of the body cavity, the bending section 21 bent to the first angle θ1 receives an external force acting in the second direction that decreases the bend angle, as shown in FIG. 11A.

In the external-force detection mode, the distribution of tension of the wire 16 is such that the stationary frictional force acting toward the distal end A1 at each position of the wire 16 has vanished, as shown in FIG. 10B. Therefore, when an external force acts in a direction that bends the bending section 21 in the second direction, the tension measured by the tension sensor 35a disposed at the proximal end of the wire 16 immediately changes, as shown in FIG. 11B.

The processor 41 monitors a variation in the tension measured by the tension sensor 35a after entering the external-force detection mode (step S3), and performs external-force reduction control for reducing the external force (step S4) if the variation exceeds a predetermined threshold value.

Examples of the external-force reduction control include a method involving cutting off the power to the motor 34 to set the bending section 21 in a freely movable state and a method involving driving the wire 16 in a direction for actively decreasing the bend angle. The driving method in the direction for actively decreasing the bend angle may include a method involving driving the driving device 30 so as to return to the origin point where the tension of the wire 16 is zero or to set a tension difference between wires 16 forming a pair to zero.

Accordingly, with the medical system 100, the control device 40, and the control method according to this embodiment, the external force applied to the distal end of the insertion section 11, which is flexible, can be detected more quickly and accurately at the proximal end of the insertion section 11, and the external-force reduction control can be performed before the load applied to the bending section 21 increases due to the external force. Consequently, for example, when the endoscope 10 is to be pulled out, the bending section 21 or biological tissue that comes into contact therewith can be prevented from being damaged.

Although the description of this embodiment relates to a case where an external force that bends the bending section 21 in the second direction is to be detected, the embodiment can also be applied to a case where an external force that further bends the bending section 21 in the first direction is to be detected.

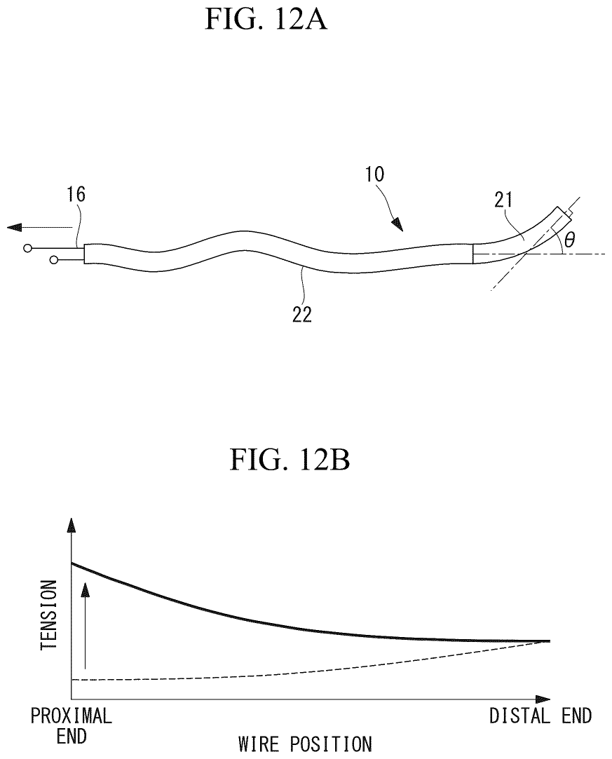

For example, if the wire 16 is stationary in the distribution of tension indicated with the solid line in FIG. 10B, the processor 41 inputs a drive amount D2 of the wire 16 and a tension T2 of the wire 16 into the model in the memory 42, so as to acquire the drive amount ΔD of the wire 16 corresponding to the dead zone (step S1). The processor 41 drives the motor 34 by the acquired drive amount ΔD of the wire 16, so as to pull the wire 16 (step S2). Accordingly, the bending section 21 moves to the first position P1 in FIG. 7. An example of the shape of the endoscope 10 in this state is as shown in FIG. 12A, and the distribution of tension of the wire 16 in the lengthwise direction of the endoscope 10 is as shown in FIG. 12B.

If the wire 16 is stationary in the distribution of tension indicated with the solid line in FIG. 10B (dashed line in FIG. 12B), the stationary frictional force acting toward the distal end at each position of the wire 16 has vanished. However, even when an external force acts in the first direction onto the distal end of the bending section 21 to increase the bend angle, an external force acting in a direction for further loosening the wire 16 is not transmitted to the tension sensor 35a at the proximal end of the wire 16 in a state where the wire 16 is loose, so that a change in the tension is not detected.

On the other hand, if the wire 16 is stationary in the distribution of tension indicated with a solid line in FIG. 12B, a stationary frictional force acting toward the distal end at each position of the wire 16 has occurred. Therefore, when an external force acts in the first direction onto the distal end of the bending section 21 to increase the bend angle in this state, the external force is immediately transmitted to the tension sensor 35a at the proximal end of the wire 16, so that the change in the tension can be detected with high sensitivity.

According to this embodiment, in order to achieve the distribution of tension in FIG. 12B, the wire 16 is pulled by the drive amount ΔD of the wire 16 corresponding to the dead zone, so that the bend angle of the bending section 21 remains to be maintained at the first angle θ1. Therefore, the bent state of the bending section 21 of the endoscope 10 does not change, so that an endoscopic image displayed on the display device 70 does not have to be changed.

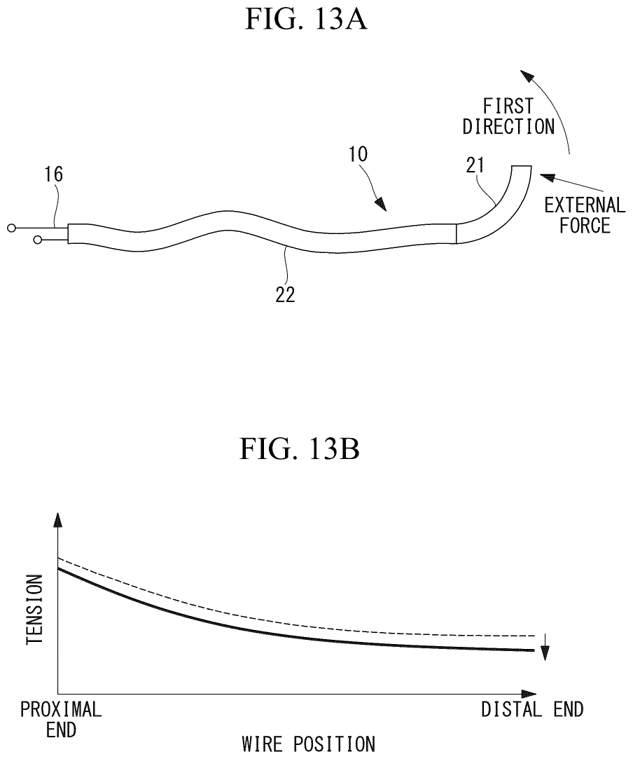

When the insertion section 11 is further pushed into the body in this state, the bending section 21 bent to the first angle θ1 receives, from the inner wall of the body cavity in contact therewith, an external force in the first direction that increases the bend angle, as shown in FIG. 13A.

In the external-force detection mode, the distribution of tension of the wire 16 is such that a stationary frictional force acting toward the distal end at each position of the wire 16 has occurred, as indicated with a dashed line in FIG. 13B. Therefore, when an external force acts in a direction that bends the bending section 21 in the first direction, the tension measured by the tension sensor 35a disposed at the proximal end of the wire 16 immediately changes, as indicated with a solid line in FIG. 13B.

The processor 41 monitors a variation in the tension measured by the tension sensor 35a after entering the external-force detection mode (step S3), and performs external-force reduction control for reducing the external force (step S4) if the variation exceeds a predetermined threshold value.

Examples of the external-force reduction control include a method involving cutting off the power to the motor 34 to set the bending section 21 in a freely movable state and a method involving driving the wire 16 in a direction for actively increasing the bend angle.

Accordingly, with the medical system 100, the control device 40, and the control method according to this embodiment, the external force applied to the distal end of the insertion section 11, which is flexible, can be detected more quickly and accurately at the proximal end of the insertion section 11, and the external-force reduction control can be performed before the load applied to the bending section 21 increases due to the external force. Consequently, for example, when the endoscope 10 is to be inserted, biological tissue that comes into contact with the bending section 21 or the bending section 21 can be prevented from being damaged.

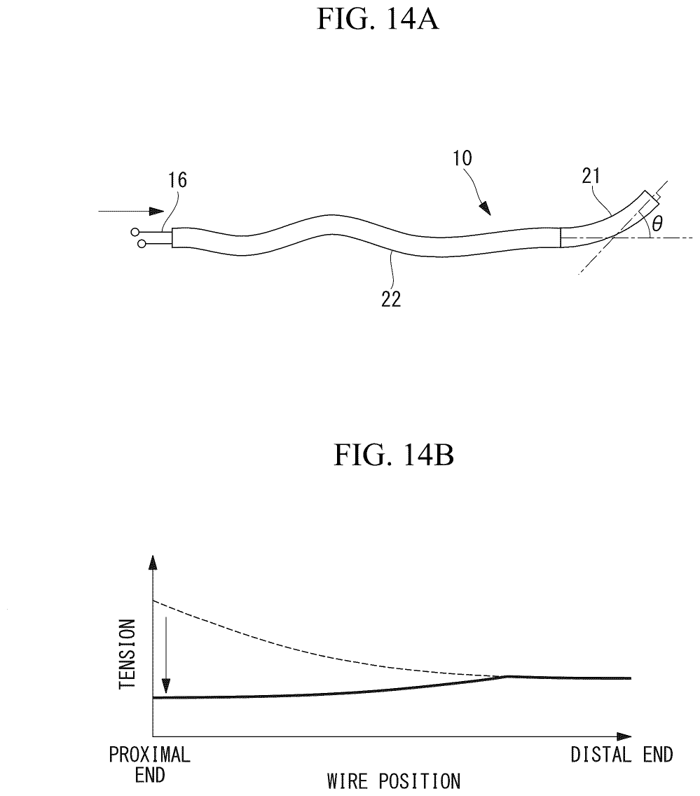

In this embodiment, the distribution of tension of the wire 16 shown in FIG. 10B in which the wire 16 is loosened is achieved from the distribution of tension of the wire shown in FIG. 8B in which the wire 16 is pulled to set the bend angle of the bending section 21 to the first angle θ1 to immediately before the bend angle of the bending section 21 starts to change from the first angle θ1. Similarly, the distribution of tension of the wire indicated with the solid line in FIG. 12B in which the wire 16 is pulled is achieved from the distribution of tension of the wire 16 indicated with the dashed line in FIG. 12B in which the wire 16 is loosened to immediately before the bend angle of the bending section 21 starts to change from the first angle θ1. Alternatively, as shown in FIGS. 14A and 14B, the wire 16 may be loosened or pulled slightly beforehand, that is, by a drive amount smaller than the drive amount ΔD of the wire corresponding to the dead zone, instead of immediately before the bend angle of the bending section 21 starts to change from the first angle θ1.

Accordingly, the external-force detection performance deteriorates with decreasing drive amount, as compared with the case where the wire 16 corresponding to the dead zone is loosened or pulled by the drive amount ΔD. However, this is advantageous in that, after entering the external-force detection mode, the bending section 21 can start moving better upon re-entering the operation mode by the operator, such as a doctor.

Next, the medical system 100, the control device 40, and the control method according to a second embodiment of the present invention will be described with reference to the drawings.

In this embodiment, sections with configurations identical to those in the first embodiment described above will be given the same reference signs, and descriptions thereof will be omitted.

The medical system 100 according to this embodiment differs from the first embodiment in that, for example, each wire attachable-detachable section 23′ and each wire driver 31′ independently drive a pair of wires 16a and 16b for bending the first bending section 21a upward and downward.

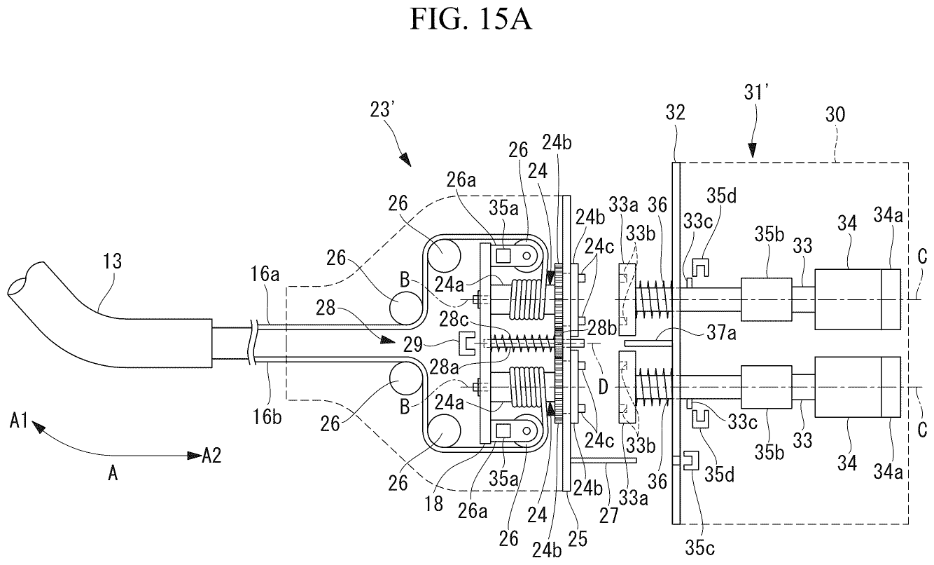

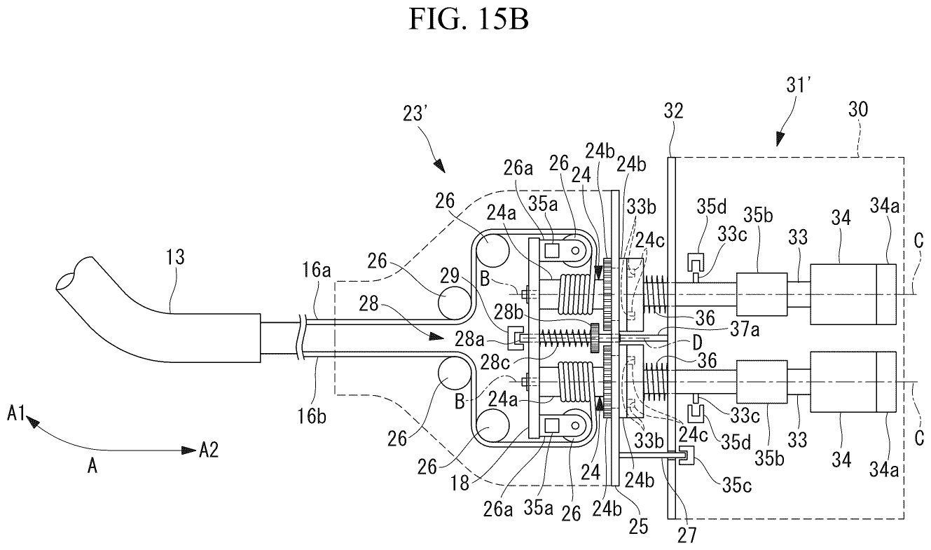

FIGS. 15A and 15B illustrate the configuration of the wire attachable-detachable section 23′ and the wire driver 31′. FIG. 15A illustrates the wire attachable-detachable section 23′ and the wire driver 31′ in a state where they are separated from each other, and FIG. 15B illustrates the wire attachable-detachable section 23′ and the wire driver 31′ in a state where they are connected to each other. For example, FIGS. 15A and 15B illustrate the wire attachable-detachable section 23′ including the pair of wires 16a and 16b for bending the first bending section 21a upward and downward, and also illustrate the wire driver 31′. The remaining wire attachable-detachable sections 23′ and the remaining wire drivers 31′ also have the configurations in FIGS. 15A and 15B.

Each wire attachable-detachable section 23′ has a pair of rotating drums 24 and a coupling mechanism 28 that couples the pair of rotating drums 24 to each other.

Each rotating drum 24 is supported by the support member 25 in such a manner as to be rotatable around the rotation axis B extending in the longitudinal direction A of the insertion section 11. Each rotating drum 24 has the winding pulley 24a disposed coaxially with the rotation axis B, and also has a gear 24d fixed to the winding pulley 24a and disposed coaxially with the rotation axis B.

The proximal end of each of the wires 16a and 16b is guided to the winding pulley 24a via at least one pulley 26, and is wound around the winding pulley 24a. Each rotating drum 24 rotates around the rotation axis B so that the corresponding wire 16a or 16b is pulled or fed. The gear 24d is a spur gear that rotates integrally with the winding pulley 24a.

In a state where the wire attachable-detachable section 23′ is separated from the wire driver 31′, the coupling mechanism 28 limits the rotation of the pair of rotating drums 24 to prevent the pair of wires 16a and 16b from loosening. The coupling mechanism 28 has a cylindrical member 28a, a link gear 28b, and an elastic member 28c.

The cylindrical member 28a is supported by the support member 25 in such a manner as to be rotatable around the rotation axis C extending in the longitudinal direction A and extendable and retractable in the longitudinal direction. The rotation axis C is parallel to the rotation axis B of each rotating drum 24. The proximal end of the cylindrical member 28a extends through the support member 25 and protrudes outward of the wire attachable-detachable section 23′ so as to be exposed at the proximal end A2 of the wire attachable-detachable section 23′.

The link gear 28b is a spur gear fixed to the cylindrical member 28a and disposed coaxially with the rotation axis C.

The elastic member 28c is, for example, a spring and biases the link gear 28b and the cylindrical member 28a toward the proximal end A2.

As shown in FIG. 15A, in the state where the wire attachable-detachable section 23′ is separated from the wire driver 31′, the link gear 28b and the cylindrical member 28a that are biased by the elastic member 28c are disposed at a separated position. The link gear 28b at the separated position is disposed between a pair of gears 24d and meshes with both of the pair of gears 24d. As a result, the pair of rotating drums 24 rotate in conjunction with each other in opposite directions from each other, thus causing the pair of wires 16a and 16b to be pulled or fed in conjunction with each other as if the pair of wires are looped (looped state). In the looped state, if the bending section 21 bends upward or downward due to an external force, the pair of wires 16a and 16b do not loosen, and the relationship between the rotational angles of the rotating drums 24 and the bend angle of the bending section 21 is maintained.

In contrast, as shown in FIG. 15B, in the state where the wire attachable-detachable section 23′ is connected to the wire driver 31′, the cylindrical member 28a is pressed toward the distal end A1 by an engagement member 37a (to be described later) against the biasing force of the elastic member 28c, so that the link gear 28b and the cylindrical member 28a are disposed at a connected position. The link gear 28b disposed at the connected position does not mesh with the pair of gears 24d. As a result, the pair of rotating drums 24 do not rotate in conjunction with each other, thus causing the pair of wires 16a and 16b to be pulled or fed independently of each other (countervailing state). Reference sign 29 denotes a countervailing-state sensor that detects the cylindrical member 28a during the countervailing state.

The wire driver 31′ has a pair of shafts 33 and a pair of motors (power generators) 34 respectively connected to the pair of shafts 33.

Each shaft 33 is supported by the support member 32 in such a manner as to be rotatable around the rotation axis C and extendable and retractable in the longitudinal direction A. The rotation axis C is the central axis of each shaft 33 and is aligned with the rotation axis B of the corresponding rotating drum 24 in a state where the attachable-detachable section 14 is connected to the driving device 30.

Each motor 34 generates a rotational force as a driving force in accordance with electric power supplied from the power source, and rotates the corresponding shaft 33 around the rotation axis C. For the respective motors 34, the wire driver 31′ is provided with two encoders 34a that detect the rotational speeds and the rotational angles of the motors 34. Each encoder 34a is connected to the proximal end of the corresponding motor 34.

The support member 32 is provided with the engagement member 37a for releasing the coupled state between the pair of rotating drums 24 by the coupling mechanism 28. The engagement member 37a is a cylindrical member exposed at the distal end of the wire driver 31′ and is provided at a position corresponding to the cylindrical member 28a. As shown in FIG. 15B, in the state where the wire attachable-detachable section 23′ is connected to the wire driver 31′, the engagement member 37a presses the cylindrical member 28a to the connected position.

The wire driver 31′ has a coupling 33a as a mechanism that is provided at each of the pair of shafts 33 and that couples each motor 34 to the corresponding rotating drum 24.

Next, the control method executed by the control device 40 according to this embodiment will be described below.

The control device 40 enters the external-force detection mode upon receiving a predetermined trigger.

As one example, the following description relates to a case where the trigger is input to the processor 41 in a state where the bending section 21 is bent in the first direction to the first angle θ1 as a result of the operator operating the first angle knob 53 of the manipulation device 50 to increase the tension of the wire (referred to as “first wire” hereinafter) 16a, located at the inner bend of the bending section 21, of the pair of wires 16a and 16b and to sufficiently decrease the tension of the wire (referred to as “second wire” hereinafter) 16b located at the outer bend.

In this case, the drive amount of the first wire 16a is the drive amount D1, the tension of the first wire 16a is the tension T1, and the bending section 21 is located at the first position P1 in FIG. 7. The distribution of tension of the first wire 16a in the lengthwise direction of the endoscope 10 in this state is as shown in FIG. 8B, and the distribution of tension of the second wire 16b is sufficiently loosened along the entire length so as to be substantially zero.

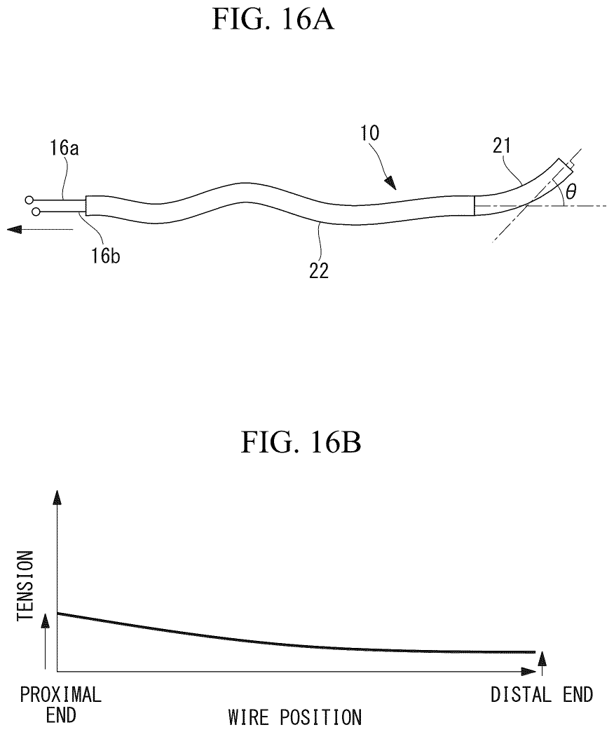

When the trigger is input in this state, the processor 41 pulls the second wire 16b alone within a range in which a bend angle θ1 of the bending section 21 is maintained, as shown in FIG. 16A, so as to generate a predetermined tension in the second wire 16b at the distal-end position of the bending section 21, as shown in FIG. 16B. The drive amount of the second wire 16b in this case may be a preset drive amount. Since a large tension is generated in the first wire 16a, the bending section 21 does not move until the tension in the second wire 16b becomes sufficiently large, and the bend angle θ1 is maintained.

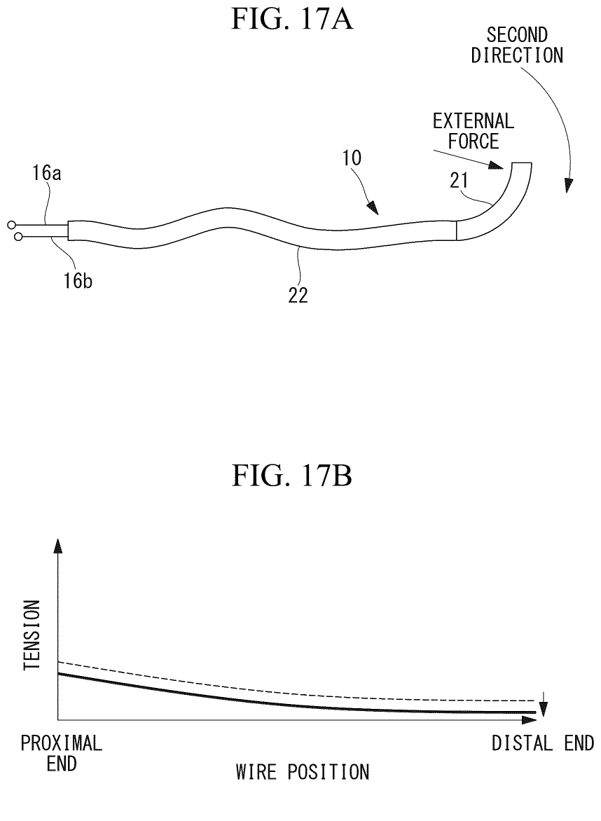

When the second wire 16b is stationary in the distribution of tension shown in FIG. 16B, a stationary frictional force is generated toward the distal end at each position of the second wire. Therefore, as shown in FIG. 17A, when an external force is applied in the second direction to the distal end of the bending section 21 to decrease the bend angle in this state, the external force is immediately transmitted to the tension sensor 35a at the proximal end of the second wire 16b, as shown in FIG. 17B, so that the change in the tension can be detected with high sensitivity.

Accordingly, for example, when the endoscope 10 is to be pulled out, biological tissue that comes into contact with the bending section 21 or the bending section 21 can be prevented from being damaged. In this case, unlike the medical system 100 and the control method according to the first embodiment, the external-force detection mode can be set without decreasing the tension of the first wire 16a. This is advantageous in that, after the external-force detection mode, the bending section 21 can start moving better upon re-entering the operation mode by the operator, such as a doctor.

Although the description of this embodiment relates to the case where an external force that bends the bending section 21 in the second direction is to be detected, the embodiment can also be applied to a case where an external force that bends the bending section 21 in the first direction is to be detected.

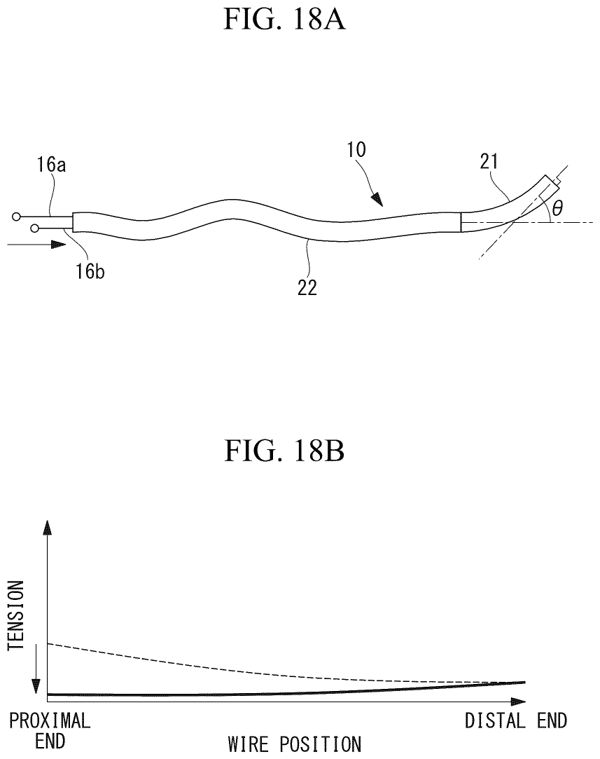

For example, when the first wire 16a is in the distribution of tension in FIG. 8B and the second wire 16b is stationary in a state where the tension thereof is substantially zero along the entire length thereof, the processor 41 first sets the distribution of tension in FIG. 16B by pulling the second wire 16b without loosening the first wire 16a, similarly to the above. Subsequently, as shown in FIG. 18A, the second wire 16b is loosened, as indicated with a solid line in FIG. 18B, in the range in which the bend angle of the bending section 21 is maintained.

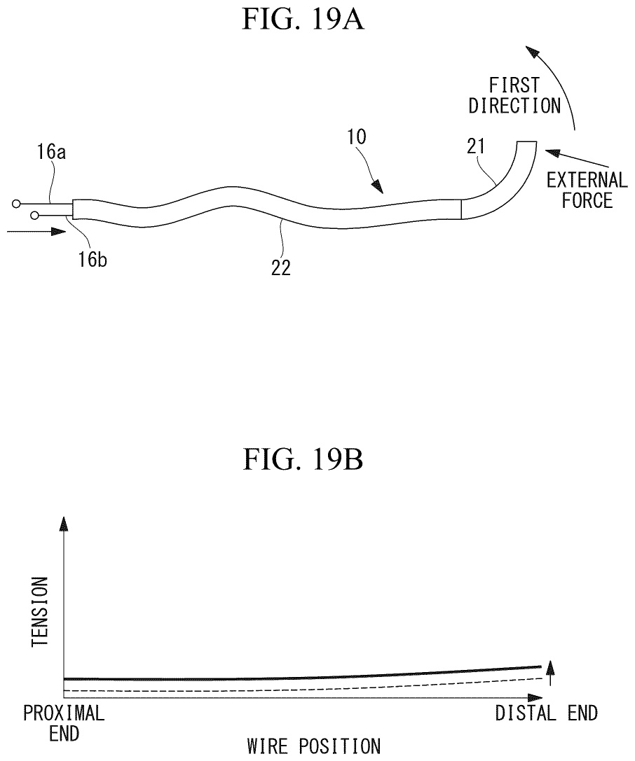

When the second wire 16b is stationary in the distribution of tension indicated with the solid line in FIG. 18B, a stationary frictional force is generated toward the proximal end A2 at each position of the second wire 16b. Therefore, as shown in FIG. 19A, when an external force is applied in the first direction to the distal end of the bending section 21 to increase the bend angle in this state, the external force is immediately transmitted to the tension sensor 35a at the proximal end of the second wire 16b, as shown in FIG. 19B, so that the change in the tension can be detected with high sensitivity.

Therefore, for example, when the endoscope 10 is to be pushed in, biological tissue that comes into contact with the bending section 21 or the bending section 21 can be prevented from being damaged. In this case, the external-force detection mode can be similarly set without decreasing the tension of the first wire 16a. This is advantageous in that, after the external-force detection mode, the bending section 21 can start moving better upon re-entering the operation mode by the operator, such as a doctor.

When the second wire 16b is pulled without loosening the first wire 16a, an external force acting in the second direction is detected with the second wire 16b and an external force acting in the first direction is detected with the first wire 16a. This is advantageous in that external forces acting in both directions can be detected.

Furthermore, when the second wire 16b is loosened within the range in which the bend angle of the bending section 21 is maintained, an external force acting in the first direction is detected with the second wire 16b, and the external force acting in the first direction is also detected with the first wire 16a, thereby enabling double detection of the external force and achieving enhanced robustness.

As an alternative to this embodiment in which the second wire 16b is pulled without loosening the first wire 16a, the second wire 16b may be pulled while loosening the first wire 16a. In this case, for example, as shown in FIG. 14B, the first wire 16a is loosened by a drive amount smaller than the drive amount ΔD of the wire 16 corresponding to the dead zone. Accordingly, even if the second wire 16b is pulled when entering the external-force detection mode, the distribution of tension of the second wire 16b can be set to the state shown in FIG. 16B without causing the bending section 21 to move.

Consequently, this is advantageous in that, after the external-force detection mode, the bending section 21 can start moving better in the second direction upon re-entering the operation mode by the operator, such as a doctor.

In this case, the distribution of tension of the first wire 16a in the external-force detection mode is as shown in FIG. 14B, and the distribution of tension of the second wire 16b is as shown in FIG. 16B. An external force acting in the second direction can be detected with both the first wire 16a and the second wire 16b. Accordingly, this is advantageous in enabling double detection of the external force acting in the second direction and achieving enhanced robustness.

Although the tension sensor 35a that measures the tension of each wire 16 is described as an example in this embodiment, the configuration is not limited to this. As an alternative to the tension sensor 35a, the torque sensor 35b that acquires a drive torque as information related to a change in the tension of the wire 16, 16a, or 16b or an electric current sensor that acquires an electric current value of the motor 34 may be employed. The acquired drive torque or the acquired electric current value may be converted into the tension of the wire 16, 16a, or 16b.

As an alternative to the endoscope 10 as a medical manipulator, another manipulator may be employed. For example, a treatment tool robot equipped with an end effector and a bending section at the distal end thereof may be employed.

The external-force reduction control is performed when the variation in the tension in the wire 16 or 16b exceeds the predetermined threshold value, and the threshold value used may be an arbitrary value. For example, in order to prevent tissue within the body cavity from being damaged, the threshold value is preferably set to 18 N or smaller.

### REFERENCE SIGNS LIST

10 endoscope (medical manipulator)
16 wire
16, 16a first wire (wire)
16, 16b second wire (wire)
21 bending section
30 driving device
35a tension sensor (sensor)
40 control device
41 processor
42 memory
100 medical system

What is claimed is:

1. A control device that controls a driving device connected to a proximal end of a flexible medical manipulator,
wherein the driving device drives at least one wire for bending a bending section at a distal end of the medical manipulator,
wherein tension of the wire is measured at the proximal end,
wherein the control device comprises at least one processor, and
wherein, in a state where a bend angle of the bending section is set to a certain angle, the processor is configured to control the driving device to pull or loosen the wire within a range in which the bend angle is maintained at the certain angle to detect an external force acting on the bending section based on a change in the tension of the wire.

2. The control device according to claim 1, wherein the range in which the bend angle is maintained at the certain angle is before the bend angle changes.

3. The control device according to claim 1,
wherein, if the change in the tension of the wire exceeds a predetermined threshold value after the wire is pulled or loosened within the range in which the bend angle is maintained at the certain angle, the processor is configured to control the driving device in an opposite direction from the change in the tension of the wire.

4. The control device according to claim 1, further comprising:
at least one memory that stores a model indicating a relationship between the bend angle and at least one of a drive amount of the wire or the tension of the wire,
wherein the processor is configured to acquire the drive amount of the wire for maintaining the bend angle at the certain angle based on the model, the bend angle, and the tension.

5. The control device according to claim 1,
wherein the state where the bend angle is set to the certain angle is a state where the wire is bent to the certain angle by being pulled, and
wherein the processor is configured to control the driving device to loosen the wire within the range in which the bend angle is maintained at the certain angle.

6. The control device according to claim 1,
wherein the state where the bend angle is set to the certain angle is a state where the wire is loosened within the range in which the bend angle is maintained at the certain angle, and
wherein the processor is configured to control the driving device to pull the wire within the range in which the bend angle is maintained at the certain angle.

7. The control device according to claim 1,
wherein the at least one wire comprises a first wire for bending the bending section in a first direction and a second wire for bending the bending section in a second direction different from the first direction, and
wherein, in a state where a bend angle of the bending section in the first direction is set to a certain angle by pulling the first wire and loosening the second wire, the processor is configured to control the driving device to pull the second wire within the range in which the bend angle of the bending section is maintained at the certain angle to detect an external force acting in the second direction on the bending section based on a change in tension of the second wire.

8. The control device according to claim 7,
wherein, if an amount of decrease in the tension of the second wire exceeds a predetermined threshold value after the second wire is pulled within the range in which the bend angle is maintained at the certain angle, the processor is configured to control the driving device in an opposite direction from a change in tension of the first wire.

9. The control device according to claim 7, wherein the range in which the bend angle is maintained at the certain angle is before the bend angle changes.

10. The control device according to claim 1,
wherein the at least one wire comprises a first wire for bending the bending section in a first direction and a second wire for bending the bending section in a second direction different from the first direction, and
wherein, in a state where the second wire is pulled within a range in which a bend angle of the bending section in the first direction is maintained at a certain angle after the bend angle of the bending section is set to the certain angle by pulling the first wire and loosening the second wire, the processor is configured to control the driving device to loosen the second wire within the range in which the bend angle is maintained at the certain angle to detect an external force acting in the first direction on the bending section based on a change in tension of the second wire.

11. The control device according to claim 10,
wherein, if an amount of increase in the tension of the second wire exceeds a predetermined threshold value after the second wire is loosened within the range in which the bend angle is maintained at the certain angle, the processor is configured to control the driving device in an opposite direction from the change in the tension of the second wire.

12. The control device according to claim 10, wherein the range in which the bend angle is maintained at the certain angle is before the bend angle changes.

13. A medical system comprising:
a flexible medical manipulator that comprises at least one wire for bending a bending section at a distal end of the medical manipulator and a sensor that measures tension of the wire at a proximal end of the medical manipulator;
a driving device that is connected to the proximal end of the medical manipulator and that drives the medical manipulator; and
a control device that controls the driving device, wherein the control device comprises at least one processor, and
wherein, in a state where a bend angle of the bending section is set to a certain angle, the processor is configured to control the driving device to pull or loosen the wire within a range in which the bend angle is maintained at the certain angle so that the sensor detects an external force acting on the bending section based on a change in the tension of the wire.

14. The medical system according to claim 13,
wherein the at least one wire comprises a first wire for bending the bending section in a first direction and a second wire for bending the bending section in a second direction different from the first direction, and
wherein, in a state where a bend angle of the bending section in the first direction is set to a certain angle by pulling the first wire and loosening the second wire, the processor is configured to control the driving device to pull the second wire within the range in which the bend angle of the bending section is maintained at the certain angle so that the sensor detects an external force acting in the second direction on the bending section based on a change in tension of the second wire.

15. The medical system according to claim 13,
wherein the at least one wire comprises a first wire for bending the bending section in a first direction and a second wire for bending the bending section in a second direction different from the first direction, and
wherein, in a state where the second wire is pulled within a range in which a bend angle of the bending section in the first direction is maintained at a certain angle after the bend angle of the bending section is set to the certain angle by pulling the first wire and loosening the second wire, the processor is configured to control the driving device to loosen the second wire within the range in which the bend angle is maintained at the certain angle so that the sensor detects an external force acting in the first direction on the bending section based on a change in tension of the second wire.

16. A control method for driving at least one wire for bending a bending section at a distal end of a flexible medical manipulator, the control method comprising:
measuring tension of the wire at a proximal end of the medical manipulator; and
performing control to pull or loosen the wire within a range in which a bend angle of the bending section is maintained at a certain angle to detect an external force acting on the bending section based on a change in the tension of the wire from a state where the bend angle is set to the certain angle.

17. The control method according to claim 16, further comprising performing control to minimize the change in the tension of the wire if a variation in the tension of the wire exceeds a predetermined threshold value after the wire is pulled or loosened within the range in which the bend angle is maintained at the certain angle.

18. The control method according to claim 16,
wherein the at least one wire comprises a first wire for bending the bending section in a first direction and a second wire for bending the bending section in a second direction different from the first direction, and
wherein the performing control comprises performing, in a state where a bend angle of the bending section in the first direction is set to a certain angle by pulling the first wire and loosening the second wire, control to pull the second wire within the range in which the bend angle of the bending section is maintained at the certain angle to detect an external force acting in the second direction on the bending section based on a change in tension of the second wire.

19. The control method according to claim 16,
wherein the at least one wire comprises a first wire for bending the bending section in a first direction and a second wire for bending the bending section in a second direction different from the first direction, and
wherein the performing control comprises performing, in a state where the second wire is pulled within a range in which a bend angle of the bending section in the first direction is maintained at a certain angle after the bend angle of the bending section is set to the certain angle by pulling the first wire and loosening the second wire, control to loosen the second wire within the range in which the bend angle is maintained at the certain angle to detect an external force acting in the first direction on the bending section based on a change in tension of the second wire.
